(19) United States
(12) Patent Application Publication
(10) Pub. No.: US 2010/0312097 A1
(43) Pub. Date: Dec. 9, 2010

(54) Hybridized optical-MRI method and device for molecular dynamic monitoring of in vivo response to disease treatment

(75) Inventors: Pascal Gallant, Quebec (CA); Ozzy Mermut, Quebec (CA)

Correspondence Address: MERCHANT & GOULD PC, P.O. BOX 2903, MINNEAPOLIS, MN, 55402-0903, US

(73) Assignee: Institut National D'Optique, Quebec (CA)

(21) Appl. No.: 12/802,363

(22) Filed: Jun. 4, 2010

Related U.S. Application Data
(60) Provisional application No. 61/184,527, filed on Jun. 5, 2009.

(51) Int. Cl. A61B 5/055

(52) U.S. Cl. 600/411

(57) ABSTRACT

An apparatus for providing physiological information from an organism in disease diagnosis and treatment monitoring, for use in an MRI instrument. The apparatus operates on the concept of hybridized magneto-optical sensitivity. The MRI includes an MRI scanner and a controller for controlling the MRI scanner. The MRI scanner provides a magnetic field of at least 0.5T. The apparatus further includes a front end built of non-magnetic components, comprising light guides for illuminating a region of interest (ROI) and for collecting light emitted at said ROI; and a back-end comprising a light source for injecting light into said light guides; a light detector for receiving light collected at said ROI; and a processing and control unit for processing said light collected at said ROI.

This application claims benefit of U.S. Ser. No. 61/184,527, filed Jun. 5, 2009 and which application is incorporated herein by reference. To the extent appropriate, a claim of priority is made to the above disclosed application.

### FIELD OF THE INVENTION

The present invention relates to a method and device for molecular dynamic monitoring of in vivo response to disease treatment. More specifically, the present invention uses an MRI to do so, along with and optical module.

### BACKGROUND AND PRIOR ART

Many biomolecular and physiological processes are based on chemical reaction pathways producing radical pair intermediaries. Examples of the importance of the radical pair mechanism in biology and medicine are too numerous to mention but include many enzymatic reactions, disease action and even therapies (through the use of appropriate drugs), such as photodynamic therapy in cancer treatment. Recent theories even indicate that the aging process could be related to strong contributions of the radical pair mechanism in the cell biochemistry.

These radical pairs are sensitive to magnetic fields, affecting the energy level configurations of the intermediaries and/or products of the biochemical reactions, at the fine and hyperfine structure levels through the Zeeman effect and singlet-triplet intersystem crossing dynamics. These changes in energy level configuration affects the optical emissions that can potentially be produced during the process (either spectral signature, amplitude perturbation or time dependent properties) offering an opportunity to optically probe or control the process at close to real time.

A magneto-optic effect, MOE, refers to a perturbation of an optical emission imparted by application of a magnetic field. As illustrated in FIG. 1 (Prior art) an external magnetic field can alter the reaction rate and/or product distribution in reactions involving radical pairs (Petrov, Borisenko et al. 1994). The orientation of the electron spins of photoexcited species is important in determining their magnetic susceptibility. The spin exchange in a radical pair system, and hence the kinetics and yield of luminescence, are mainly governed by hyperfine coupling of the unpaired electrons with the magnetic moments of the nuclei and the interaction of these electrons with external magnetic fields (Ferraudi 1998; Bandyopadhyay, Sen et al. 2002). Weak magnetic fields can thus affect the photochemical and photophysical luminescence properties of a triplet state radical pair via Zeeman splitting and hyperfine coupling (Eichwald and Walleczek 1996). Typically, magnetic field strengths of <100 mT, or about 15 to 30 times smaller than the field strength of a typical MRI unit, can induce Zeeman splitting, resulting in lifting the degeneracy of the triplet electronic states (T₀ and T₊₁, T₋₁). The consequence of this is an alteration of the rate of intersystem crossing (ISC) and modified production of reactive radicals. Perturbations in the hyperfine coupling manifest as changes in the rate of ISC due to the interaction between the magnetic field and the nuclear spins of the radical pair (Nath and Chowdhury 1984; Petrov, Borisenko et al. 1994).

The electron spin of the radical pair determines whether the pair is in a singlet or triplet configuration. Radical pairs produced from singlet recombinations will often react to form stable products on a very short time-scale (<1 ns) and are not susceptible to magnetic field effects on optical emissions (Scaiano, Cozens et al. 1994). On the other hand, triplet radical pairs are much longer-lived species and are more likely to be affected by a weak external magnetic field.

Nielsen et al., US 2008/0230715 A1 describes how to use spatially inhomogeneous weak magnetic fields (in the few hundreds of mT) with an optical molecular contrast agent described simply as a “donor-acceptor” complex to enhance optical molecular imaging, in a similar fashion to photoacoustic tissue imaging. The magnetic field inhomogeneity affects the donor-acceptor complex by modifying its singlet-triplet population ratio, modifying the fluorescence-to-phosphorescence ratio at a spatially-localized point of the subject under study. This, in essence, circumvents the impact of scattering on the optical signal and potentially enables high spatial resolution diffuse optical tomography. By modifying the spatial profile of the magnetic field, one can scan the subject under study to provide a whole tomographic dataset. Nielsen et al. specifically mentions several times that the apparatus extracts “structural” information. However, Nielsen et al. do not teach how to use the magneto-optical technique as a mean to capture physiological information.

Long, U.S. Pat. No. 7,519,411 B2 describes how magnetic fields can be used to affect reaction dynamics of photosensitive compounds in the context of cancer photodynamic therapy. It is mentioned that fluorescence-to-phosphorescence ratios can be used as indicator of the favoured chemical reaction pathway of PDT (Type I or Type II). The Type II pathway is highly favoured in an oxygen-rich environment, while the Type I is favoured in hypoxic regions. The Type I pathway is based on the radical pair mechanism and thus sensitive to magnetic field effects. The optical signal is thus affected differently by the magnetic field in each case and this difference can be linked to the environmental nature of the photoreactive process (in this case, local concentration of molecular oxygen). Long never specifically mentions the use of weak magnetic fields, but does mention ranges of B-field sensitivity of a number of reaction types such as triplet-triplet annihilation in strong fields (˜7T), uncharged radical pairs sensitivity to weak or medium fields (<0.5 T) and charged anion-cation radical pairs in weak fields (˜0.01T).

### SUMMARY OF THE INVENTION

It is an object of the present invention to provide a method and apparatus to use magneto-optical information in an imaging concept integrating an optical device inside a standard MRI scanner to provide physiological information in disease diagnosis and treatment monitoring.

In accordance with one aspect of the invention, there is provided a method of hybridizing magnetic and optical fields for providing physiological imaging of an organism. The method comprises the steps of:
(a) generating a magnetic field with an MRI device;
(b) generating an optical field with an optical device integrated within the MRI device;
(c) providing a magneto-optically sensitive contrast marker, wherein the hybridization of both magnetic and optical fields is provided by said magneto-optically sensitive contrast marker injected into the organism
   wherein said hybridization is based on the local production of paramagnetic radical pairs from the contrast marker interacting with the organism's tissue; and
(d) detecting a magnetic resonance response from standard MRI techniques; and
(e) detecting at least one of absorbance, luminescence, fluorescence or phosphorescence generated by the interaction of the contrast marker with the organism's tissues.

In accordance with another aspect of the invention, there is provided an apparatus for providing physiological information from an organism in disease diagnosis and treatment monitoring, for use in an MRI instrument, the apparatus operates on the concept of hybridized magneto-optical sensitivity. The MRI includes an MRI scanner and a controller for controlling the MRI scanner. The MRI scanner provides a magnetic field of at least 0.5T. The apparatus further includes a front end built of non-magnetic components, comprising light guides for illuminating a region of interest (ROI) and for collecting light emitted at said ROI; and a back-end comprising a light source for injecting light into said light guides; a light detector for receiving light collected at said ROI; and a processing and control unit for processing said light collected at said ROI.

### BRIEF DESCRIPTION OF THE DRAWINGS

The present invention will be better understood after having read a description of a preferred embodiment thereof, made in reference to the following drawings, in which:

FIG. 1 illustrates that the origin of magneto-optic effects, MOE, in PSs arise from: (A) the Zeeman splitting of degenerate states, T₀, T₊₁, T₋₁, in response to increasing B-field; and (B) the hyperfine coupling, hfc, between donor-acceptor (D-A) singlet and triplet states;

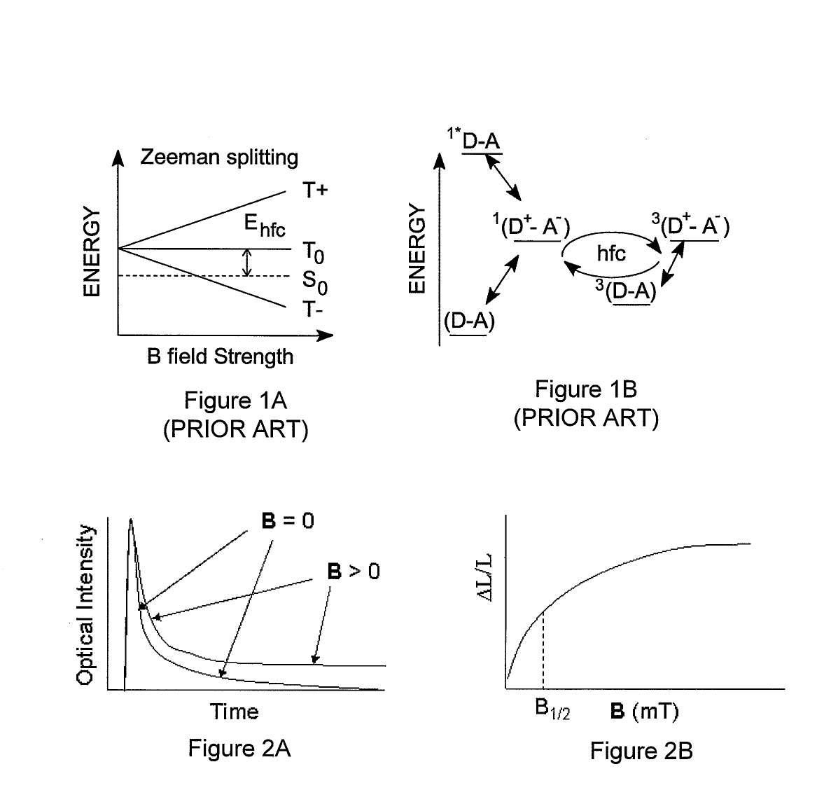

FIG. 2 illustrates the magneto optical photodynamics showing theoretical variation at B=0 and B>0 of A) the MOPS emission decay curve and B) the optical density;

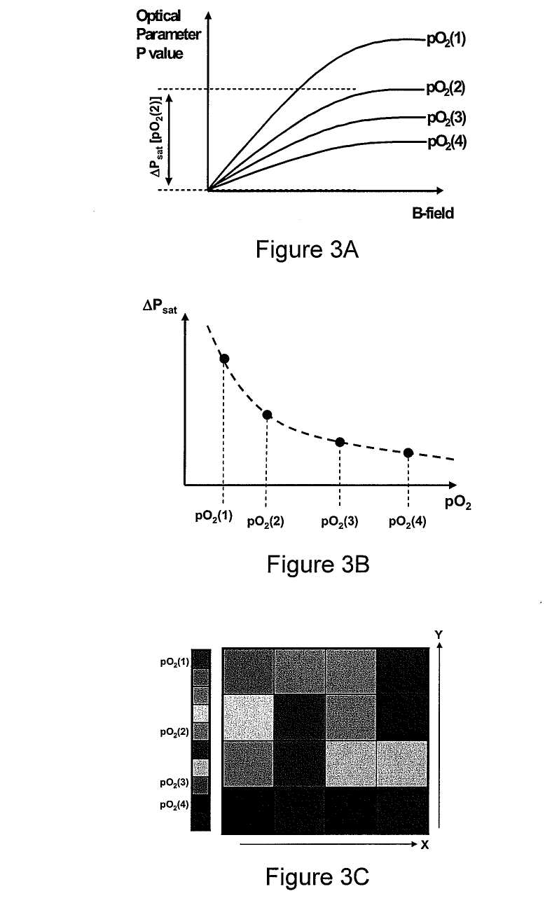

FIG. 3 illustrates the process of building a 2D topographic map of the pO₂ physiological parameter by MOD. A) Acquisition of the MO response curves for different pO₂ values and selection of a criterion to map the optical parameter P to the pO₂ value. In this particular example, the saturation value of the optical parameter at high B-field is used. B) Building of the calibration curve of the criterion vs. pO₂. C) 2D mapping of pO₂ in false colors based on the selected criterion calibration established in (B);

FIG. 4 is an illustration of A) Two major pathways of cytotoxic response in PDT. Type II generates singlet oxygen. Type I generates radicals and radical oxides that can be affected by weak magnetic fields. Radical pairs that are sensitive to B-fields can be generated when a photosensitizer, PS, initially reacts with a non-oxygen reactant, R, and eventually generates reactive oxygen species (i.e. oxide radicals). The rate constants for singlet state fluorescence, triplet state phosphorescence, intersystem crossing, PDT, hydrogen abstraction and electron transfer are represented by k_{S}, k_{T}, k_{ISC}, k_{PDT}, k_{HA} and k_{ET} respectively. B) Outline of photochemical steps involved in the two PDT pathways;

FIG. 5 is a schematic representation of the proposed overall scheme for the preferred embodiment of a hybridized optical-MRI apparatus; and

FIG. 6 are schematic representations of the preferred embodiment of the optical device add-on to the MRI scanner described in the present invention. A) Diagram of the 2D prototype optical add-on to be integrated in the MRI scanner. A number of optical fibers built in a 2D array forms the front-end of the device to probe the specimen within the MRI scanner magnetic field. The fibers are used to deliver the laser light and collect the optical signal and transfer it to the back-end of the device in the MRI scanner control room. A xy scanner is used as the fiber selector to send laser light and collect the signal. Raster scanning the array produces a final 2D mapping of the optical data collected from the specimen. B) The various alternatives of interaction between the optical 2D prototype front end with a specimen. i) Non-contact configuration. Both source and collection are done point-by-point. ii) In-contact configuration. The array mount is made flexible to match the specimen topology. iii) An alternative non-contact method where the whole specimen is illuminated at once through a dedicated fiber channel for the laser light. Signal collection is done point-by-point in a raster scan fashion.

### DESCRIPTION OF A PREFERRED EMBODIMENT OF THE INVENTION

The present invention concerns the use of magneto-optical effects to probe or monitor a biochemical/physiological process in vivo. This has been demonstrated in the prior art, in the case of photodynamic therapy, using a straightforward system combining a highly sensitive optical device using weak magnetic fields (less than 500 mT). The potential of the technique for PDT and other medical treatment applications combined with the now ubiquitous availability of MRI in clinical environments and micro-MRI in preclinical laboratories offers the possibility of a relatively simple hybridized optical-MRI device to be developed and used, based on magneto-optic effect occurring in a strong magnetic field (typically greater than 1 T). Furthermore, an MRI can operate in various field modulation modes, providing more complex time-varying magnetic fields configurations than basic static fields.

Typically, the MRI scanner is used to establish diagnostic and follow therapy effectiveness through morphology of tissues. Therapy monitoring in this case is dependent on the tissue structure in the MRI dataset. For example, in cancer, treatments will be monitored by looking at the tumour size, tissue cellular characteristic (necrotic, haemorrhaging, amount of stroma, etc.) and blood perfusion, through functional MRI.

The present invention thus proposes the use of hybridized magneto-optic effects produced from an MRI instrument to invoke changes in the optical emission intensity, lifetime, and spectral splitting of a fluorescent or phosphorescent signal from an optically-sensitive drug or other biocompatible compound. The preferred embodiment is an optical apparatus embedded in an MRI platform intended and designed to generate and detect magneto-optic effects from within the strong (on the order of 0.7 to 3 T) magnetic field of the parent MRI construct. This enables near real-time tracking of the photo-induced chemical, physical, kinetic or pharmaceutical response of the injected compound through the magneto-optic effect, to monitor the treatment progress or efficiency or both. This result provides information on the status of the treatment providing feedback that the end-user can act upon (i.e. make a decision to change dosing parameters or change the other therapeutic modalities). For example, in the case of a photodynamic drug, dynamic information about local tissue oxygenation levels is required in order to optimize the photo toxicity treatment program closer to real time, or indicate a critical time-point for switching to ionizing radiation therapy or antiangiogenic therapy. This is a current issue in PDT, currently hindering its wider scale use in the clinical field.

It is of note that the invention proposes to use the magnetic field of the MRI and the optical signal from the compound in a synergistic fashion to evaluate physiology. This is different to Nielsen's goal of using an inhomogeneous magnetic field to select a particular optical signal value spatially and extract structural information, thereby using the magnetic field to improve instrumental performance and enhance optical data. Although the compound can be designed as a targeting optical contrast agent, Nielsen does not describe probing physiology with the combination of the magnetic and optical fields.

The present invention makes use of an optically-activated drug or other biocompatible compound that reemits luminescence and that produces radical pairs according to the biochemical environment characteristics. This optically-activated molecule can also associate to a free radical naturally present in the tissue to form a radical pair, assuming favourable conditions exist (adapted molecular structure of the photo activated compound, presence of the target free radical in sufficient concentration locally, etc.).

The optical device add-on allows optical activation of a drug compound within the patient and subsequent detection of luminescence from the drug from within the MRI scanner. The luminescence signal can be described by a number of “optical parameters”, e.g. luminescence intensity, lifetime, spectral properties, spectral band shape, etc. By looking at variations of one or a combination of these parameters as the magnetic field is changed provides the information on physiology as a means to monitor the state of a disease or treatment.

The variation of the optical parameter as a function of the B-field strength is defined as the magneto-optical response. Nielsen does present such curves in his patent but limits them to fluorescence intensity. in contrast, the present invention teaches to look at the variation of the entire magneto-optical response curve as a function of a specific physiologically-relevant parameter like, but not limited to, pO₂ (local oxygen concentration in the tissues). This is different from Nielsen who teaches the use of the fluorescence-to-phosphorescence intensity ratio or half-life (lifetime) ratio as a “processing filter” to spatially select the relevant photons. In the case of the present invention, the technique uses a measurement of the optical parameter of choice (e.g. fluorescence lifetime) for at least two values of the magnetic field.

Changes in the magneto-optical response curve can be extracted from a number of processing techniques such as: difference between MO effect saturation at high fields and values at B=0, slope of the variation of the optical parameter at a specific B-field value vs the physiological parameter value, or other.

Multipoint measurements of the optical parameter can allow building a spatial map of the physiological parameter. Combining this with the MRI dataset can allow adaptation of the technique to 3D tomography, using appropriate reconstruction algorithms, where the MRI dataset can be used as a priori information.

In summary, a process for implementing measurements according to one embodiment of the invention can be summarized as follows.

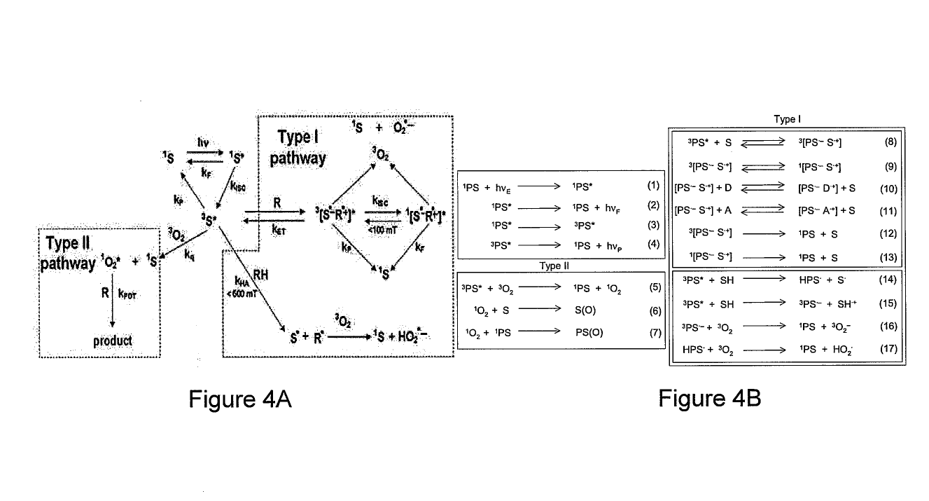

1) Establish the magneto-optical response curve for a number of the specific physiologically-relevant parameter values (here we use local oxygen concentration in tissues, pO₂, see FIG. 3 left).

2) use a criterion of measurement to distinguish the physiology parameterized response curve (here we use the B-field saturation value of the selected optical parameter relative to the absence of field value, delta-Pₛₐₜ, FIG. 3 center).

3) Map the correspondence of delta-Pₛₐₜ vs pO₂(x) for each measurement point. This produces a 2D distribution map (an image, FIG. 3 right) of the physiological parameter. Note that this is extensible to 3D in a tomographic setup.

To build the magneto-optical response curve for various values of the physiologically-relevant parameter, one can characterize the photoactivated compound into a separate measurement apparatus using a variable low-field magnet, similar to the apparatus described by Long. Alternatively, one can use magnetic shielding of some sort with the MRI scanner, use a low field scanner or use the MRI scanner fringe field, or a combination thereof.

Conceptualization of the measurement process includes the following steps:
1. Characterize the photoactivated luminescent compound magneto optical response
2. Establish the physiological-to-optical parameter criterion to use for mapping (e.g. delta-Pₛₐₜ vs pO₂(x) in previous point)
3. Measure the optical signal in the MRI scanner appropriately, according to the physiological-to-optical parameter criterion chosen (e.g. for delta-Pₛₐₜ, two measurements are needed, one outside of the MRI scanner at B=0 T and one with the subject fully into the MRI bore at maximum field where saturation of the magneto optical effect will occur). Data acquisition outside of the bore of the MRI scanner (within the so-called fringe magnetic field of the scanner) may be used to provide optical data in weak B-field ranges.
4. Extract the physiologically-relevant parameter distribution map from the measurements based on points 1 and 2.

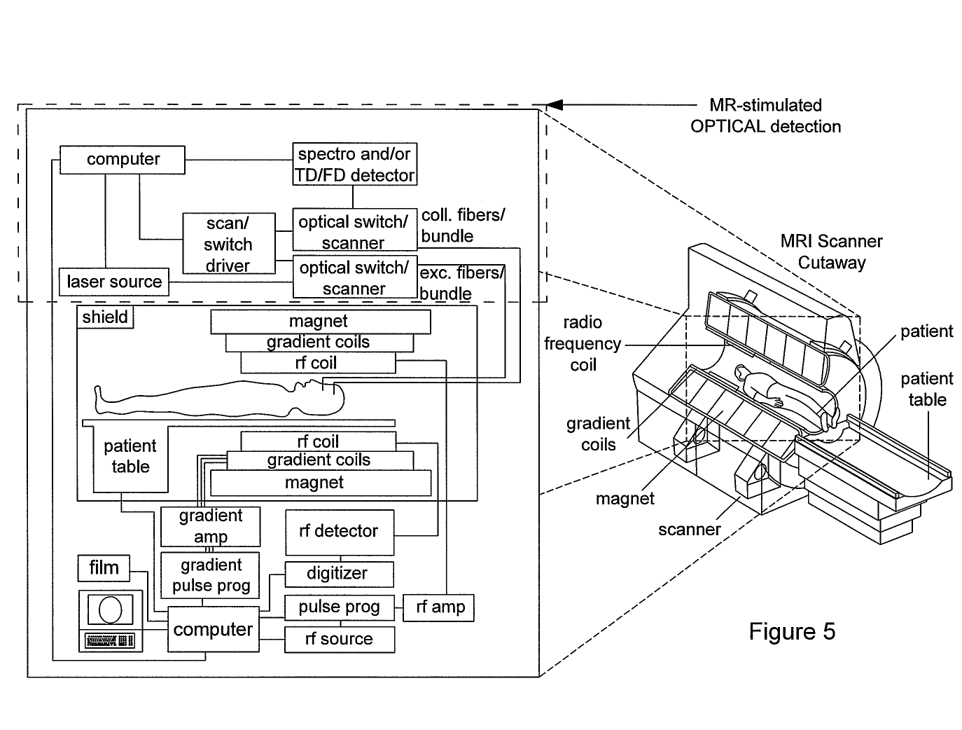

Photodynamic Therapy (PDT) is a good example of a potential application of this concept. While PDT offers very good promise as a targeted cancer treatment modality, many attempts to use PDT in the clinic have been hindered by the complex dosimetry problem (particularly in deep tissues), a lack of an accepted definition of dose, and a suitable technique to measure/monitor doses in vivo. As explained by Long, PDT operates by two oxygen-dependent pathways that lead to photo toxicity in tumour cells (FIG. 4, Rosenthal and Ben Hur 1995). The Type II pathway is thought to be dominant in most PDT and occurs when molecular oxygen is converted to cytotoxic singlet oxygen via energy transfer (e.g. donating an electron or accepting a proton) from the excited triplet state photosensitizer compound. In equilibrium with pathway II is Type I photosensitization, which involves charge transfer or hydrogen atom transfer reactions with triplet state photosensitizers. Since oxygen rapidly quenches the excited triplet state of the photosensitizer, the Type I pathway is more significant at low oxygen concentrations (i.e. in poorly vascularised tissues) or in polar environments (Allen, Sharman et al. 2001). Because the Type I pathway is based on the radical pair mechanism, it is sensitive to magnetic fields. The balance between pathways of Type I and Type II is dependent on local oxygenation of the cancer tissue and can be monitored through the changes of the magnetically affected optical signal.

While MOEs have been explored in a variety of model photo induced charge transfer systems (i.e. donor-acceptor complexes), the phenomenon had, until recently, not been well-realized for any biomedical application (Bhattacharyya and Chowdhury 1993; Petrov, Borisenko et al. 1994). Currently, the concept of using such magneto-optical effects has been demonstrated in model biological systems in vitro (cell phantoms). (Mermut, Noiseux et al. 2008; Noiseux, Mermut et al. 2008; Mermut, Diamond et al. 2009).

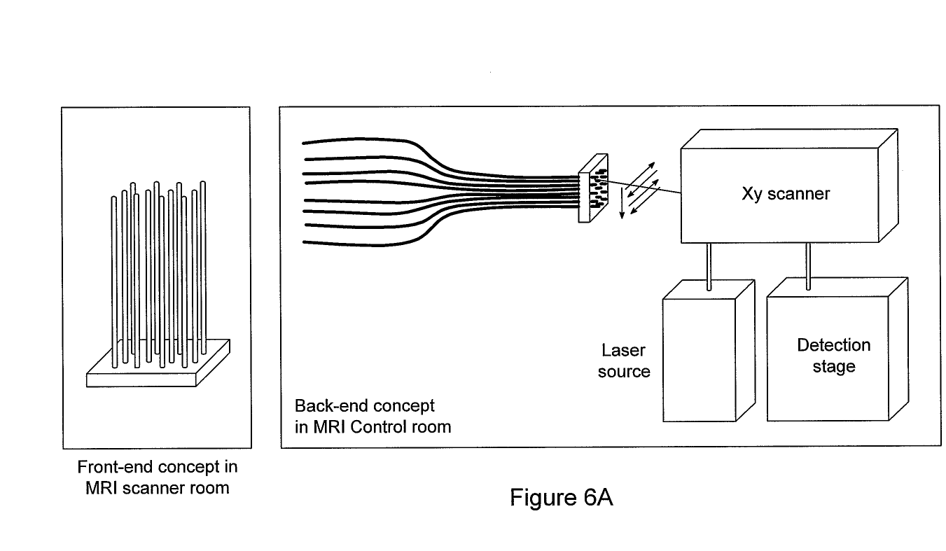

The present invention thus concerns an apparatus for carrying out the process described above. In a preferred embodiment, the invention more specifically concerns an optical device add-on to a standard MRI scanner (FIG. 5). Indeed, one of the objects of the invention is to maximize the existing infrastructure in clinical settings. MRI machines are now widely distributed, and the invention helps further capitalize on the existing technology to refine both diagnostic and treatment applications of MRI machines.

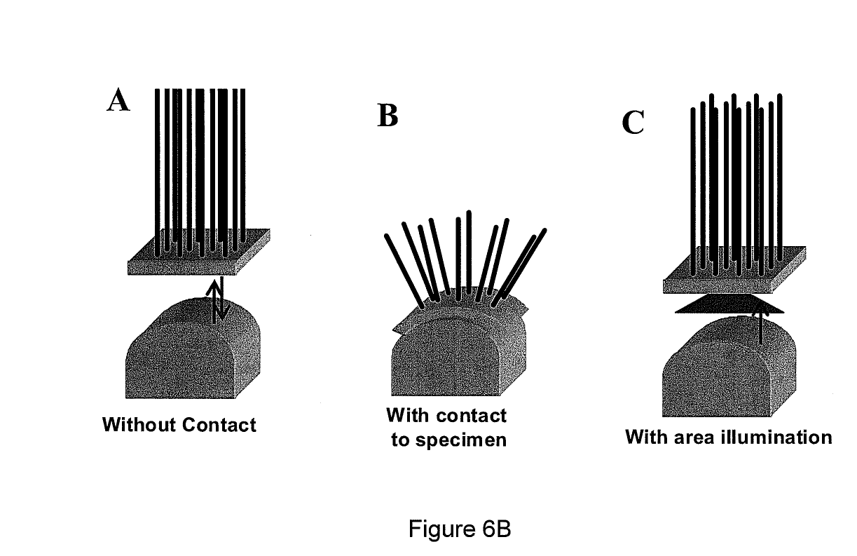

What follows is a description of a preferred embodiment of the apparatus according to the invention. A person skilled in the art will appreciate that this description is not limitative, and further refinements, additions and modifications can be effected without departing from the basic principles of the present invention.
1) The apparatus is built into two parts, a front-end that is magnetically insensitive and thus compatible to fit into an operational MRI scanner, and a back-end optical and electronic equipment containing optical sources and detectors, data acquisition and recording hardware, that can be integrated into a MRI scanner control room (FIG. 6A).
2) The MRI scanner, as is currently well known, provides a static field rated at 0.5 T or higher.
3) The apparatus front end and back-end are connected by non-magnetically built light-guides, such as optical fibers or fiber bundles.
4) The light guides serve both as a delivery mechanism for the illumination wavelength and the collection of the light to the detection system.
5) The front end can be designed for non-contact observation of the specimen, using bulk optics such as objective lenses and mirrors, a fiber bundle coupled to an objective lens or a number of individual optical fibers positioned into a rectangular or circular array (FIG. 6B left). Such a design provides a 2-D spatial image of an area of interest of the scanned subject, with pixel values referencing an optical parameter value of interest as per the described magneto-optical technique, be it fluorescence intensity, lifetime, spectral band intensity or any parameter thereof that is affected by the magneto-optical principle.
6) The non-contact configuration can enable 3D tomography if the front-end is mounted on a rotating gantry that is insulated from the magnetic field and RF interferences produced by the operating MRI scanner. This permits capture of multiple images of the subject in sequence that allows tomography when coupled to the MRI dataset and an appropriate reconstruction algorithm (as is known in the art).
7) Alternatively, the front-end can be designed for in-contact acquisition, whereas a number of fiber optics cables or fiber bundles are positioned in contact to the scanned subject, enabling 2D proximity optical imaging of the subject surface (FIG. 6B center).
8) The in-contact configuration can enable 3D optical tomography when the optical dataset is coupled to the MRI dataset and an appropriate reconstruction algorithm (as is known in the art).
9) The back-end illumination source can be a cw, intensity-modulated or pulsed laser.
10) The laser source is point-scanned on the proximal end of the delivery light guide assembly, providing a point illumination of the subject. That point of illumination is raster-scanned on the subject surface at the distal end according to the selected light-guide input by the back-end scanning apparatus.
11) Alternatively, a full field illumination of the entire area of interest on the subject can be done using a dedicated delivery light guide for the light source (FIG. 6B right).
12) The back-end detection side can make use of full-field or area detectors using spatially resolved sensors, including but not limited to, CCD cameras, intensified CCDs, gated CCDs, modulated MCP-built intensifiers, APD arrays, etc.
13) Alternatively, the back-end detection side can be built using raster scanning techniques for the illumination source, the detector field of view or both. The detection system can be frequency-domain based (modulation, and phase detection), time-domain based (photon counting) or spectrally resolved.
14) Each pixel can contain raw information such as, but not limited to, a spectrum, a time-resolved optical signal, a modulated signal or an intensity value.
15) The back-end is coupled to a processing and control unit that is synchronized with the MRI scanner control unit for operation and acquisition of the optical data.

Advantageously, the following hardware and software can further be used with the present invention:
(1) Time and spectrally resolved system using hardware/components related to (2) or (3)
(2) Time-domain, spectrally resolved system using: i) pulsed light source(s) (LEDs, laser diodes, or supercontinuum lasers with suitable drivers); ii) photon counting detector, iii) some way to spectrally resolve the emitted signal (i.e. spectrometer)
(3) Frequency domain system using i) intensity modulated light sources (LEDs, laser diodes, supercontinuum) with a device for modulation such as an acousto-optic or electro-optic modulator), ii) a source to supply rf (such as rf generator), iii) a modulable detector i.e. PMT or APD.
(4) Some software that controls and sequences the magnetic modulation with the optical excitation and collection.

1. A method of hybridizing magnetic and optical fields for providing physiological imaging of an organism, said method comprising the steps of:
(a) generating a the magnetic field with an MRI device;
(b) generating an optical field with an optical device integrated within the MRI device;
(c) providing a magneto-optically sensitive contrast marker, wherein the hybridization of both magnetic and optical fields is provided by said magneto-optically sensitive contrast marker injected into the organism
wherein said hybridization is based on the local production of paramagnetic radical pairs from the contrast marker interacting with the organism's tissue; and
(d) detecting a magnetic resonance response from standard MRI techniques; and
(e) detecting at least one of absorbance, luminescence, fluorescence or phosphorescence generated by the interaction of the contrast marker with the organism's tissues.

2. The method of claim 1 wherein the contrast marker interacting with the tissues within the magnetic and optical fields generates a different and specific magneto-optical response for an optical parameter for at least two different values of the magnetic field, such as to generate a magneto-optical response curve.

3. The method of claim 1 wherein a change of the magneto-optical response curve is linked to a change in a physiological parameter of the organism tissues.

4. The method of claim 1 wherein the optical device includes an image generator to generate images.

5. The method of claim 4, wherein an image pixel represents the value of a physiological parameter of the organism tissues, based on the magneto-optical response.

6. The method of claim 1 where in the optical device is integrated in such a way to provide multiple image projections, enabling 3D tomographic imaging.

7. The method of claim 2, wherein said optical parameter is at least one of intensity, spectral properties or lifetime of the detected optical signal.

8. An apparatus for providing physiological information from an organism in disease diagnosis and treatment monitoring, for use in an MRI instrument, said apparatus operating on the concept of hybridized magneto-optical sensitivity;
said MRI including an MRI scanner and a controller for controlling said MRI scanner, said MRI scanner providing a magnetic field of at least 0.5T;
said apparatus comprising:
a front end built of non-magnetic components, comprising light guides for illuminating a region of interest (ROI) and for collecting light emitted at said ROI;
a back-end comprising:
a light source for injecting light into said light guides;
a light detector for receiving light collected at said ROI; and
a processing and control unit for processing said light collected at said ROI.

9. An apparatus according to claim 8, wherein said front end is adapted to observe said ROI with no contact.

10. An apparatus according to claim 8, wherein said front end is further provided with bulk optics.

11. An apparatus according to claim 10, wherein said bulk optics include lenses, mirrors, a fiber bundle coupled to an objective lens, a plurality of individual fibers positioned into a circular or rectangular array, or a combination thereof.

12. An apparatus according to claim 8, wherein said front end is mounted on a rotating gantry in order to capture multiple images in sequence.

13. An apparatus according to claim 8, wherein said front end is adapted to in-contact acquisition of light.

14. An apparatus according to claim 8, wherein said light source includes a cw, intensity modulated or pulsed light source.

15. An apparatus according to claim 8, wherein said light source includes a laser, a LED or any spectrally-controlled light emitting element.

16. An apparatus according to claim 15, wherein said spectrally-controlled light emitting element is a filtered arc lamp or a light bulb.

17. An apparatus according to claim 15, wherein said light is point-scanned on a proximal end of said light guides, in order to provide a point illumination of said ROI.

18. An apparatus according to claim 17, wherein said point illumination is raster-scanned on said ROI at a distal end of said light guide.

19. An apparatus according to claim 8, wherein said light guide is a dedicated delivery light guide for said light source.

20. An apparatus according to claim 8, wherein said detector includes sensors for sensing said light, said sensors including CCD cameras, intensified CCDs, gated CCDs, modulated MCP-built intensifiers, photomultiplier tubes, photon counters and APD arrays.

21. An apparatus according to claim 8, wherein said detector includes a spectral dispersion element.

22. An apparatus according to claim 8, wherein said processing and control unit is coupled to an MRI scanner control unit and synchronized therewith.

23. An apparatus according to claim 8, wherein said processing and control unit generates a magneto-optical response curve for each measurement point from the detected optical signal and generated magnetic field.

24. An apparatus according to claim 8, wherein said processing and control unit converts the measured magneto-optical response curve at each measurement point into a physiological parameter value.

25. An apparatus according to claim 8, wherein said processing and control unit generates an image from recovered physiological values by mapping said values onto spatial location of each measurement.

26. An apparatus according to claim 8, wherein said processing and control unit generates a combined optical-MRI image or tomographic image set of the organism.
